(19) United States
(12) Patent Application Publication
(10) Pub. No.: US 2017/0362631 A1
(43) Pub. Date: Dec. 21, 2017

(54) METHOD AND DEVICE FOR DETERMINING THE PRESENCE OF A MICRO-ORGANISM IN STOOLS WITH ACTIVATED CARBON PRETREATMENT

(71) Applicant: BIOMÉRIEUX, Marcy L'Etoile (FR)

(72) Inventors: Hélène BRIAND, L'Arbresle (FR); Anne DURAFFOURG COLLOMB, Saint Didier De Formans (FR)

(73) Assignee: BIOMÉRIEUX, Marcy L'Etoile (FR)

(21) Appl. No.: 15/534,112

(22) PCT Filed: Dec. 16, 2015
(86) PCT No.: PCT/FR2015/053535

§ 371 Date: Jun. 8, 2017

(30) Foreign Application Priority Data
Dec. 16, 2014 (FR) 1462481

(51) Int. Cl. C12Q 1/04; B01L 3/00; G01N 33/543

(52) U.S. Cl. CPC C12Q 1/04; G01N 33/54386; B01L 3/5023; B01L 2200/16

(57) ABSTRACT

The invention provides a determination method for determining the presence of a target microorganism in a patient from a sample of said patient's stools, the method being characterized in that it comprises the following operations:
obtaining a sample of liquid stools of said patient or a liquid sample obtained from stools of said patient, referred to as the liquid sample;
pretreating the liquid sample with activated carbon; and
using immunochromatography to detect in the resulting pretreated liquid sample the possible presence of at least one antigen of the target microorganism so as to come to a conclusion about the presence or the absence of the target microorganism in said patient.

The invention also provides a device (1) for detecting an antigen of a target microorganism in the liquid sample (3) by immunochromatography, the device including a zone (20) for purification with activated carbon (21).

The invention relates to the technical field of immunoassays and in particular of immunoassays adapted to detecting pathogens present in a patient, when the presence of pathogens can be revealed using said patient's stools. More precisely, the invention relates to a method of detecting at least one antigen of a microorganism in a liquid stool sample or in a sample obtained from a patient's stools, and it also relates to an immunochromatography detector device adapted to perform such a method.

Diarrheas represent a major health problem worldwide. In developed countries, the economic impact of gastroenteritis is considerable and the medical expenses needed to treat it represent a financial cost that is very high. Such infections lead to considerable morbidity and represent one of the major grounds for medical consultations. In unfavored countries, very frequent gastroenteritis persists throughout the year, leading to very high mortality. Populations without access to potable water are the most severely affected by this disease.

The agents responsible for gastroenteritis may be bacterial, viral, or parasitic pathogens. An examples of such pathogens, mention may be made of the following microorganisms: Clostridum difficile, Salmonella, Shigella, rotavirus, norovirus, adenovirus, Entamoeba histolytica.

Norovirus, which is particularly resistant to environmental factors, is more and more frequently involved in episodes of diarrhea. This virus is present both in industrialized countries and in developing countries, and it is implicated in gastroenteritis epidemics involving all segments of the population. According to the bibliographic study by M. M. Patel (M. M. Patel et al., Systematic literature review of role of norovirus in sporadic gastroenteritis. Emerg Infect Dis. 2008 August; 14(8): 1224-31), norovirus in children under five years of age living in industrialized countries is responsible for 900,000 medical visits, with no fewer than 64,000 cases requiring hospitalization. In developing countries, a million children are affected and the number of deaths comes close to 200,000 deaths per year. In hospitalized children under five years of age, it is, after rotavirus, the second most frequent pathogenic agent implicated in gastroenteritis (M. Lorrot, et al., Epidemiology and clinical features of gastroenteritis in hospitalized children: prospective survey during 2-year period in a Parisian hospital, France. Eur J Clin Microbiol Infect Dis. 2011 March; 30(3); 361-8).

When there are signs of gastroenteritis, it is therefore necessary to have tests that make it possible to identify the pathogenic agent(s) in order to provide the appropriate medical response quickly. The agents responsible for gastroenteritis are often transmitted by feces.

Since the symptoms last only a few days, there is no point in searching for antibodies since the immune response is too late. In order to carry out routine diagnosis, proposals have been made for immunochromatographic techniques (also known as lateral flow immunoassay techniques), that are based on searching in fecal samples for the virus and/or for at least one analyte representative of the presence of the virus. Those techniques are easy to perform, and they give a response quickly: the result can be obtained within 15 minutes (min) from performing the assay protocol. A study carried out by K. Ambert-Balay and P. Pothier, in November 2012 presents the state of the art concerning immunochromatographic tests for norovirus that are available on the market: “Evaluation of four immunochromatographic tests for rapid detection of norovirus in fecal samples” J. Clin. Virol 2013 March; 56(3): 194-8. The sensitivity results for various commercial tests listed in that publication are summarized below:

| | Rida © | Immunocard | | SD |
|---|---|---|---|---|
| | Quick | STAT © | NOROTOP © | BIOLINE |
| Sensitivity | 52% | 35% | 51% | 41% |
| study | | | | |
| results, | | | | |
| responses to | | | | |
| genogroup I + | | | | |
| genogroup | | | | |
| II | | | | |

These results show that immunochromatographic tests sold for detecting norovirus present sensitivities that are low.

Stools are media that are very complex, including the presence of commensal bacteria of the intestinal flora, of epithelial cells, of residues of digestion, . . . . When attempting to improve the sensitivity of detection, that means that it is necessary to treat stool samples prior to testing them in order to detect the pathogen(s) associated with diarrhea, and to do so in such a manner as to retain compounds present in stools that might interfere with such detection.

The method that has been used until now consists in centrifuging prior to the detection step, possibly in association with carbon pretreatment of stool samples, as described by P. H. Dennehy et al., 1994, Journal of Clinical Microbiology, 32(3): 825-827, with the VIDAS® Rotavirus test, in which the centrifuging step is essential. Because that method makes use of a centrifuge and of trained laboratory staff, it is unsuitable for use by staff outside medical analysis laboratories, such as doctors, nurses, pediatricians, staff of retirement homes or of geriatric centers, and in particular those that use immunochromatographic devices. Furthermore, even with qualified personnel, centrifuging is a step that generates cost and that consumes time. Unfortunately, given that bouts of gastroenteritis are phenomena of short duration, it is important to be able to act as quickly as possible in order to detect the pathogen(s) responsible for the pathology, firstly in order to provide the appropriate therapeutic response as quickly as possible, and secondly in order to avoid inter-human contamination. Specifically, these pathogens are transmitted very easily, which raises problems of contamination in premises having large populations, such as retirement homes, geriatric centers, nurseries, . . . .

In this context, the invention proposes a method and a device that, against all expectations, make it possible to detect at least one antigen of a microorganism, and that are thus suitable for determining the presence of said antigen in a patient's stools, in such a manner as to make it possible quickly and without expensive and constraining centrifuging equipment to conclude whether said patient is infected by a pathogen responsible for gastroenteritis, while also guaranteeing satisfactory sensitivity.

The invention provides a determination method for determining the presence of a target microorganism in a patient from a sample of said patient's stools, the method being characterized in that it comprises the following operations:
obtaining a sample of liquid stools of said patient or a liquid sample obtained from stools of said patient, referred to as the liquid sample;
pretreating the liquid sample with activated carbon; and
using immunochromatography, also known as lateral flow immunoassay, with an immunochromatography device having a sample application zone, a marking zone, and a reaction zone, to detect from the resulting pretreated liquid sample the possible presence of at least one antigen of the target microorganism so as to come to a conclusion about the presence or the absence of the target microorganism in said patient.

In the context of the invention, it has been found, unexpectedly, that putting activated carbon directly into the presence of a liquid sample of stools or with a liquid sample obtained from a patient's stools, makes it possible to deal with interferences from compounds that are naturally present in the samples, without requiring, against all expectation, a constraining step of centrifuging, thus facilitating subsequent analysis by immunochromatography to detect antigen(s) of the target microorganism. This is very important in order to be able to benefit from all of the advantages of analyses by immunochromatography, which are referred to as rapid tests, thus requiring a minimum of human and technological constraints and making such analyses adaptable to all real conditions. The invention thus makes it possible to detect in a patient an infection by a target microorganism having antigens that are to be found in said patient's stools in the event of infection.

The term “sample of liquid stools” is used to mean directly a patient's stools when they are sufficiently liquid.

The term “liquid sample obtained from stools” is used to mean either a liquid sample comprising the patient's stools and a liquid diluant, in particular a dilution buffer, or a liquid sample obtained from the patient's stools and a liquid diluant, in particular a dilution buffer, after eliminating the stools. Under such circumstances, the stools are placed in a liquid diluant, in particular a dilution buffer, thus making it possible to extract the stools, the microorganisms that might be present, the antigens of the target microorganism that is to be detected, and other components, subsequently making it possible to characterize the presence or absence of antigens of the target microorganism in the resulting liquid sample, and thus to be able to come to a conclusion about the presence or the absence of said microorganism in the patient from whom the stools originate. The quantity of stools per dilution buffer volume preferably lies in the range 5 milligrams per milliliter (mg/mL) to 50 mg/mL of dilution buffer, more preferably in the range 10 mg/mL to 30 mg/mL, still more preferably 14 mg/mL to 17 mg/mL, with 15 mg/mL and 16 mg/mL being preferred.

The dilution buffer present in the liquid sample generally contains a buffer base, a denatured charge protein, and a detergent. As an example of a buffer base, mention may be made of a phosphate buffer, a tris-HCl buffer. As an example of a denatured charge protein, mention may be made of casein, ovalbumin, and bovine serum albumin. As a detergent, mention may be made of ionic detergents such as Triton™ X100 and Tween® 20.

The method of the invention may thus comprise preparing a liquid sample from stools, by incorporating stools in a liquid diluant, in particular a dilution buffer, and preferably a dilution buffer as described above and more preferably with the quantities of stools per volume of dilution buffer as mentioned above. The stools are usually eliminated from the liquid sample after, before, or during the pretreatment of the liquid sample with activated carbon, depending on the technique used for the pretreatment.

Below, the term “liquid sample” is used to cover equally well a sample of liquid stools and a liquid sample obtained from said patient's stools. The liquid sample contains at least one antigen of the target microorganism that is to be detected.

The characterization of the liquid sample that makes it possible to come to the conclusion that an antigen of a target microorganism is present in said liquid sample is based on detecting the presence of one or more antigens of said microorganism and consequently makes it possible to conclude whether said antigen of the target microorganism is or is not present in said stools, and thus make it possible to conclude whether the patient from whom said stools come is or is not contaminated by the target microorganism. The target microorganism itself may be present or absent in the stools and thus in the liquid sample. This is of little importance since the looked-for information is to determine whether or not the microorganism is present in the patient, and for that purpose a search is made for the presence of at least one antigen of the target microorganism.

In the context of the invention, detection relies on at least one antigen of the target microorganism. Said at least one antigen may be a protein secreted in the stools (a “free” antigen) or it may be an antigen present in the structure of the microorganism, in particular a surface antigen of the microorganism. Such an antigen may be a protein, a sugar (a polysaccharide), or a lipopolysaccharide. Regardless of whether it is a free antigen or an antigen in the structure of the target microorganism that is detected, when detection is positive, it should be concluded that the patient is infected by the target microorganism, and conversely, when detection is negative, it should be concluded that the patient is not infected by the target microorganism.

The antigens of the target microorganism may be directly accessible for detection: this applies in particular when they are secreted by the microorganism or when they are to be found on the surface of the microorganism. In the event of the target antigen(s) not being directly accessible for detection (intracellular antigen, in particular for bacteria), a preparatory step of making the antigens of the target microorganism accessible for detection may be applied to the liquid sample or while it is being prepared, using techniques that are well known to the person skilled in the art and often specific to each targeted antigen. As a general rule, this preparatory step takes place prior to the pretreatment with activated carbon, however it is also possible to perform it simultaneously with the pretreatment with activated carbon. For example, extracting toxins A and B from Clostridum difficile requires little more than dilution in a phosphate-buffer saline (PBS) type buffer, whereas extracting Galactomannan from Aspergillus fumigatus requires a step of chelation in the presence of ethylene diamine tetra-acetic acid (EDTA) followed by denaturing by heating to 95° C. for 5 min. Depending on the targeted antigen and on the structural complexity of the microorganism that produces it, the person skilled in the art will combine detergents, denaturants, chaotropic agents, and possibly physical means (heating, mechanical actions such as grinding), in order to destroy structures of the microorganism and make the targeted antigen accessible for detection, taking care to preserve its immunological reactivity. When detecting viruses, the surface antigens are directly accessible and such a preparatory step is generally not necessary.

Activated carbon is porous amorphous carbon made up mainly of carbon atoms and presenting a very large specific surface area, giving it very high adsorbing power. Such carbons are generally obtained after a step of high temperature carbonization.

In the context of the invention, the pretreatment with activated carbon makes it possible to adsorb the interfering agents contained in the liquid sample in order to able subsequently to analyze the sample of stools, in particular by immunochromatography. The pretreatment with activated carbon makes it possible to make the epitopes of the antigens that are to be detected more easily accessible to the binding partners used for detection. The step of detecting said at least one antigen in the method of the invention is performed by immunochromatography, also known as lateral flow immunoassay. Tests of the lateral flow immunoassay type are also referred to as rapid tests, and they often make use of a device in the form of strips placed in a box. Such devices comprise a sample application zone, a marking zone, and a reaction zone, as described below. For more details about these types of method and device suitable for use in the context of the invention, reference may be made in particular to the following applications: WO 2004/003559; WO 2006/092103; WO 2007/081330; US 2004/0161859; WO 2012/172232; and WO 2008/018073. Rapid tests are usually performed without a washing step, in contrast to what is done during immunoassay on microplates or using the VIDAS® device sold by the Applicant. The pretreatment with activated carbon proposed in the context of the invention thus has a greater impact on the diagnostic performance of such methods and devices for lateral flow immunoassay.

In general, the pretreatment with activated carbon includes putting the liquid sample into contact with activated carbon and then separating the activated carbon and the liquid sample. Putting the liquid sample into contact with the activated carbon can be done simply, e.g. by mixing the liquid sample with activated carbon or by passing the liquid sample over activated carbon. The contact needs to be sufficient to enable the activated carbon to perform its function and eliminate agents that might interfere in subsequent detection.

In the context of the invention, the pretreatment of the liquid sample is preferably performed with a quantity of activated carbon per liter of liquid sample lying in the range 3 grams per liter (g/L) to 30 g/L, preferably in the range 6 g/L to 18 g/L, more preferably in the range 8.4 g/mL to 10.2 g/mL, with a range of 9 g/mL to 9.6 g/mL being preferred.

Advantageously, after the pretreatment, the activated carbon and the liquid sample are separated. Such separation may be performed in particular by filtering, without requiring any centrifuging step. The separation may take place simultaneously with putting into contact, in particular when the activated carbon is positioned directly on a filter system through which the liquid sample is to pass.

The pretreatment may be performed while obtaining the liquid sample. Under such circumstances, the method of the invention also includes preparing the liquid sample, and the pretreatment with activated carbon is performed on the liquid sample directly while it is being prepared, by mixing together stools, a liquid diluant, in particular a dilution buffer corresponding in particular to the above description, and the activated carbon, followed by separation enabling the liquid sample to be recovered. The activated carbon that is used may be in the from of a suspension in a liquid diluant (corresponding in particular to the above description of the dilution buffer) and should then be mixed directly with the stools, as obtained from the patient. The liquid sample obtained after separation thus no longer contains stools nor activated carbon. The separation may be performed by filtering, in particular by using a filter positioned in a sampling device.

The pretreatment may also be performed by mixing the liquid sample with activated carbon and then separating so as to recover the liquid sample. Once more, the activated carbon incorporated in the liquid sample may be in the form of a suspension in a liquid diluant (corresponding in particular to the above description of the dilution buffer). The liquid sample obtained after separation thus no longer contains activated carbon. Separation may be performed by filtering, in particular by using a filter positioned in a sampling device or directly in the application zone of the immunoassay device, and in particular of the lateral flow immunoassay device.

The pretreatment may also be performed by passing the liquid sample over activated carbon. Under such circumstances, it is possible to make provision for the liquid sample to pass over activated carbon positioned in a sampling device or directly on activated carbon positioned on the immunoassay device such as a lateral flow immunoassay device, as described in detail below. Either way, the activated carbon is retained and/or held in the device (sampling device or immunoassay device) so as to prevent it from being entrained together with the liquid sample, which could subsequently impede detection/display and reading of the results. In particular, it is possible to deposit activated carbon on a filter or on a portion of the diffusion medium used in the immunoassay device such as a lateral flow immunoassay device. Such deposition may be performed by impregnating the filter or a portion of the diffusion medium with a liquid such as the above-described dilution buffer that contains activated carbon in suspension, followed by drying.

In general manner, the liquid sample is put into contact with activated carbon prior to depositing the liquid sample on the immunoassay device such as a lateral flow immunoassay device, or directly on such an immunoassay device, the liquid sample deposited in the immunoassay device preferably being subjected to a step of eliminating stools prior to being deposited. This step may coincide with eliminating the activated carbon, if the liquid sample is put into contact with activated carbon prior to depositing the liquid sample on the immunoassay device.

The activated carbon may be in the form of activated carbon particles or activated carbon fibers, and in particular particles or fibers constituted exclusively of activated carbon, without any coating. Depending on the nature and the moment of the pretreatment that is performed, the particles or fibers of activated carbon may be in suspension in a solution, e.g. in a liquid diluant or a buffer, or they may be incorporated in a natural or synthetic porous material such as a material of the non-woven type, based on cellulose fibers or on glass fibers. When the particles of activated carbon are incorporated in a portion of the diffusion medium of an immunochromatography detection device, the organization of the material should be adapted so as to enable the activated carbon to be retained, with the carbon absorbing the interfering agents and other impurities while allowing the antigen(s) for detection to pass.

By way of example, the pretreatment with activated carbon may be performed using activated carbon in powder form. According to the classification of the American Society for Testing and Materials (ATSM), activated carbons in powder form are constituted by particles where 95%/100% of the particles are capable of passing through a screen having a mesh of size 80 US mesh (i.e. about 177 micrometers (μm)), which corresponds to particles having a size of 80 US mesh. The activated carbon that is used preferably has a particle size in the range 100 US mesh to 400 US mesh, more preferably in the range 140 US mesh to 270 US mesh, and still more preferably 200 US mesh. By way of example, mention may be made of Norit® CN1 activated carbon, which has particles of a size lying in the range 140 US mesh to 270 US mesh.

The size of particles of activated carbon is determined by screening using techniques known to the person skilled in the art. An example protocol is described in the D2862-10 ASTM standard “standard test method for particle size distribution of granular activated carbon”.

The activated carbon may also be in the form of fibers having a diameter lying in the range 2 μm to 50 μm.

The activated carbon that is used preferably has a specific surface area greater than 500 square meters per gram (m²/g), preferably greater than 1000 m²/g, more preferably greater than 1300 m²/g. By way of example, Norit® CN1 activated carbon presents a specific surface area of 1400 m²/g.

The activated carbon should also preferably be capable of absorbing at least 10 grams (g) of methylene blue (CAS No. 61-73-4) per 100 g of carbon, and preferably at least 20 g/100 g, more preferably at least 25 g/100 g. By way of example, Norit® CN1 activated carbon is capable of absorbing 29 g/100 g.

Unlike prior techniques, in the method of the invention, the pretreatment with activated carbon is not followed by a centrifuging step, nor does the method include a centrifuging step.

The method of the invention can be used for determining whether a microorganism is or is not present in a patient, which microorganism may be selected from viruses, bacteria, and parasites, in particular of the following types: Clostridum difficile, Salmonella, Shigella, rotavirus, norovirus, adenovirus, Entamoeba histolytica, and more particularly adapted to determining the presence of a virus such as rotavirus, adenovirus, or norovirus, which is a virus of very small size.

The norovirus genus of the caliciviridae family is a non-enveloped virus, of size lying in the range 27 nanometers (nm) to 35 nm, possessing an icosahedral capside and a ribonucleic acid (RNA) genome. Because of its genetic diversity, the norovirus genus is subdivided into genogroups and then into genotypes. Protein sequencing has made it possible to define five genogroups (I to V) with genogroups I, II, and IV infecting man. As a result of analyzing amino acid sequences of the major protein of the capside, eight genotypes are now known for genogroup G1 and more than 17 genotypes for genogroup GII (D. Zheng, et al. Norovirus classification and proposed strain nomenclature. Virology 2006 Mar. 15; 346: 312-323, and extended to 19 by Weng et al. Molecular epidemiology of noroviruses in children and adults with acute gastroenteritis in Wuhan China, 2007-2010. Arch. Virol. 2012 December: 157: 2417-24). The designation adopted by the scientific community gives the genogroup GI or GII followed by the genotype GI.1, GII.4 . . . . The method of the invention is adapted to determine the presence of all of these genotypes.

In conventional manner, detecting at least one antigen of the target microorganism in the liquid sample consists in using immunoassay to detect the interaction between at least one antigen of the microorganism of interest and a binding partner of the antigen such as an antibody or an antibody fragment. In preferred manner, the antibody or antibody fragment is specific to the antigen that is to be detected. It is possible to use a monoclonal or polyclonal antibody or a plurality of monoclonal antibodies. These antibodies are obtained using techniques that are well known to the person skilled in the art.

Naturally, in the present application, the prefix “immuno” e.g. in the term “immunoassay”, should not be considered as indicating strictly that the binding partner is necessarily a partner of immunological origin, such as an antibody or an antibody fragment. Specifically, and as is well known to the person skilled in the art, this term is used more widely to designate tests and methods in which the binding partner is not a partner of immunological origin or nature, but, for example, consists in a receiver for the analyte that it is desired to detect and/or quantify. The required condition is that the binding partner in question is capable of binding with the looked-for analyte, preferably in specific manner. Thus, mention may be made of the enzyme-linked immunosorbant assay (ELISA) test, as a test that makes use of binding partners that are not strictly speaking immunological, and that may be referred to more broadly as “ligand binding assay” tests, even though the term “immuno” is included in the full term corresponding to the acronym ELISA. For reasons of clarity and uniformity, the term “immuno” is used in the present application to designate any biological analysis making use of at least one binding partner adapted to bind with the looked-for analyte and to detect and/or quantify it, preferably in specific manner, even when the binding partner is not strictly speaking of immunological nature or origin.

Examples of binding partners that are not of immunological nature or origin include nanofitins, receivers for the antigen of interest, aptamers, DARPins, or any other molecule that is known to interact with the antigen of interest.

Nanofitins (trade name) are small proteins that, like antibodies, are capable of binding with a biological target, thus enabling it to be detected, captured, or merely to be targeted within an organism.

Aptamers are oligonucleotides, generally RNA or deoxyribonucleic acid (DNA), identified in banks containing up to 10¹⁵ different sequences, by an in vitro combinatory selection method known as SELEX for “Systemic evolution of ligands by exponential enrichment” (A. D. Ellington and J. W. Szosta, 1990, Nature, 346: 818-822). Most aptamers are RNA compounds, given the ability of RNA to adopt structures that are varied and complex, which makes it possible to create cavities at its surface that are of various shapes, thus enabling various ligands to be fixed. These are biochemical tools of interest that can be used in biotechnological, diagnostic, or therapeutic applications. Their selectivity and their ligand-fixing properties are comparable to those of antibodies.

“DARPins”, i.e. Designed Ankyrin Repeat ProteINS (Y. L. Boersma and A. Plütckthun, 2011, Curr. Opin. Biotechnol. 22: 849-857) are another class of proteins capable of mimicking antibodies and of fixing on target proteins with high affinity and selectivity. They are derived from the family of ankyrin proteins, which are adaptable proteins capable of fixing the integral membrane proteins of the spectrin/actin network that constitutes the “backbone” of the cellular plasma membrane. The structure of ankyrins is based on repeating a motif of about 33 amino acids, and the same applies to DARPins. Each motif has a secondary structure of the helix-turn-helix type. DARPins contain at least three and preferably four to five repeated motifs that are obtained by “screening” combinatory banks.

Said at least one antigen of interest is detected by immunoassay, preferably by sandwich type detection, which is a technique well known to the person skilled in the art involving two binding partners of the analyte. One of the two partners may be coupled to a marker in order to form a conjugate or a tracer. The other binding partner may be captured on a solid support. The latter is then referred to as a capture partner and the former as a detection partner.

The measured signal issued during immunoassay is thus proportional to the quantity of analyte in the biological sample.

The term “marker” is used in particular to mean any molecule containing a group that is reactive with a group of the binding partner, directly without chemical modification, or after chemical modification in order to include such a group, which molecule is capable of generating a detectable signal either directly or indirectly. A non-limiting list of such direct detection markers consists in:
enzymes that produce a detectable signal, e.g. by colorimetry, fluorescence, or luminescence, such as horseradish peroxydase, alkaline prosphatase, β-galactosidase, glucose-6-phosphate dehydrogenase;
chromophores such as compounds that are fluorescent, luminescent, or staining;
radioactive molecules such as ³²P, ³⁵S, or ¹²⁵I;
fluorescent molecules such as Alexa dyes or phycocyanins; and
electrochemiluminescent salts such as organo-metallic derivatives based on acridinium or ruthenium.

It is also possible to use indirect detection systems, such as for example ligands capable of reacting with an anti-ligand. The ligand then corresponds to the marker so as to act together with the binding partner to constitute the conjugate.

Ligand and anti-ligand pairs are well known to the person skilled in the art, as applies for example to the following pairs: biotin and streptavidin; hapten and antibody: antigen and antibody; peptide and antibody; sugar and lectin; polynucleotide and complementary polynucleotide.

The anti-ligand is then detectable directly by the above-described direct detection markers, or it may itself be detectable by another ligand and anti-ligand pair, and so on.

Under certain conditions, such indirect detection systems can lead to the signal being amplified. This signal amplification technique is well known to the person skilled in the art, and reference may be made to prior patent applications FR 2 781 802 or WO 95/08000 in the name of the Applicant.

Depending on the type of marking used, the person skilled in the art will add reagents for enabling the marking or the emission of a detectable signal to be viewed by any appropriate type of measurement apparatus, such as for example: a spectrophotometer, a spectrofluorometer, a densitometer, a luminometer, or indeed a high definition camera.

The detection of at least one antigen is performed by immunochromatography, also known as lateral flow immunoassay. The devices generally used in such tests comprise a diffusion medium that enables the liquid sample to migrate, which medium is generally held on a support. Various zones are conventionally distinguished in the diffusion medium, namely an application zone for applying the liquid sample, a marking zone, and a reaction zone, which reaction zone includes a display zone (also referred to as a capture zone) and a verification or “control” zone. These various zones are in fluid-flow communication. Thus, the antigen that is to be detected, providing it is present in the sample deposited on the application zone, binds to a marked first binding partner in the marking zone, with the complex that is formed in this way then migrating to the reaction zone where it is held in the capture zone by reacting with a second binding partner that is attached to the diffusion medium, and the user can determine whether the antigen is indeed present by observing a detectable signal, as determined by the type of marker associated with the first binding partner. In general, the presence of the antigen in the sample is revealed in the form of a detectable line, commonly referred to as the test line. In general, the reaction zone also has a zone for verifying that the sample has migrated in order to inform the user that the sample has migrated correctly through the diffusion medium, upstream from the display zone. By way of example, this may be done by revealing a verification line of a predetermined color. As examples, mention may be made of the following patent applications WO 2004/003559, WO 2006/092103, WO 2007/081330, US 2004/0161859, and WO 2012/172232, which describe such devices. In particular, one such device is sold by the supplier bioMérieux under the reference VIKIA® Rota/Adeno —Ref. 31 111 for simultaneously detecting rotaviruses and adenoviruses.

The methods and devices of the invention make it possible to conclude whether at least one antigen of the microorganism of interest is or is not present in the liquid sample, and consequently in the stools of the patient in question, thus making it possible to conclude whether the patient is or is not contaminated by the microorganism, with this naturally being possible within the detection threshold limit of the method and of the device. Nevertheless, it is not excluded that the method of the invention can also be used to make a quantitative assessment of said at least one looked-for antigen and thus of the target microorganism, depending on the detection technique that is used.

The invention also provides a device for detecting at least one antigen of a microorganism in a liquid sample, the device comprising:

a) a support; and

b) a diffusion medium fixed on the support, and enabling the liquid sample to migrate, said diffusion medium comprising:
i) an application zone for applying the liquid sample;
ii) a purification zone including activated carbon;
iii) a marking zone including at least a first marked binding partner, said first binding partner being capable of binding with said at least one antigen of the microorganism that is to be detected, if present in the liquid sample, then forming a first binding partner and antigen complex; and
iv) at least one reaction zone comprising:
   a display zone for displaying the results of the detection and comprising at least one second binding partner held stationary on the diffusion medium and suitable for binding with said first binding partner and antigen complex;
   a migration verification zone enabling proper operation of the device to be verified, which zone is situated downstream from the display zone; and
   said application zone, purification zone, marking zone, and reaction zone being in fluid-flow communication to enable the liquid to diffuse.

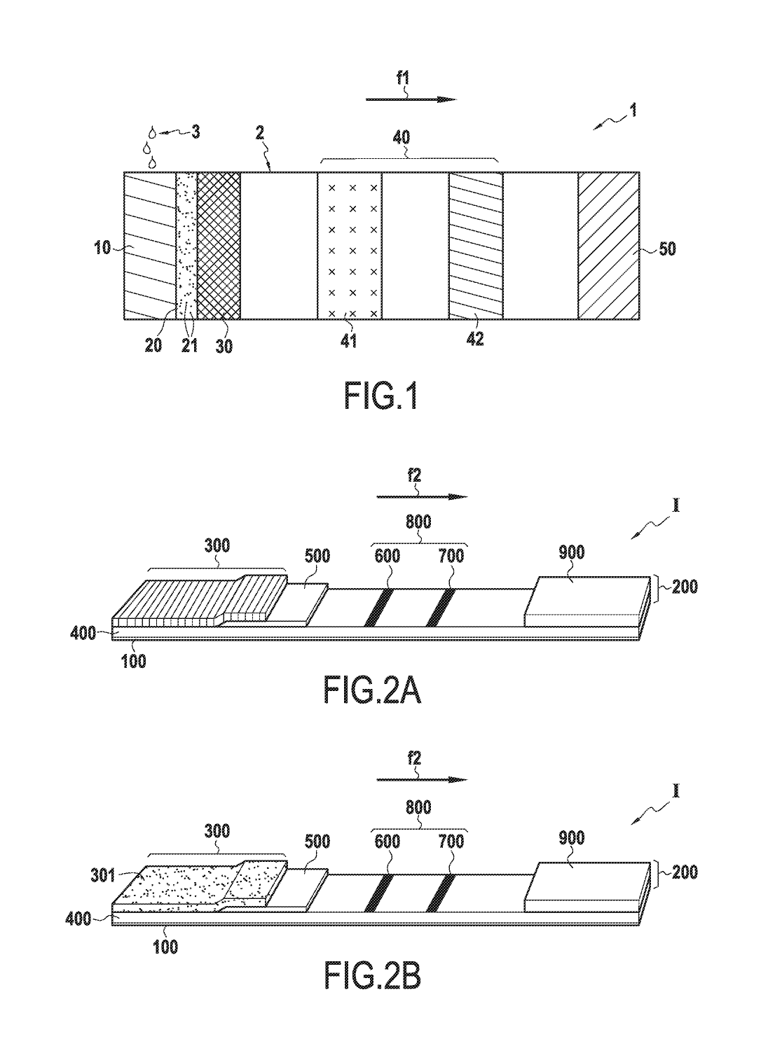

Such devices are shown in FIGS. 1 and 2B.

FIG. 1 is a diagrammatic plan view of an example of a detector device of the invention.

FIG. 2A is a diagrammatic perspective view of a prior art detector device, and FIG. 2B is a diagrammatic perspective view of an example of a detector device in accordance with the invention.

The device 1 shown in FIG. 1 enables the liquid sample to migrate in the direction f₁ and it includes a diffusion medium 2 that is fixed on a support, not shown. In known manner to the person skilled in the art, the support is water-repellant, e.g. in the form of a thin layer of plastics material. The function of the support is to stiffen, facilitate handling, and protect the diffusion medium 2. The support also serves to make the bottom face of the diffusion medium 2 waterproof and consequently to channel the flow of the sample through the diffusion medium 2.

The diffusion medium 2 has a plurality of successive zones in the direction f₁, corresponding to the direction in which the liquid sample 3 that is to be deposited will migrate: the application zone 10 for applying the liquid sample 3; the purification zone 20 having activated carbon 21; the marking zone 30 having at least a first marked binding partner; the display zone 41; and the migration verification zone 42, these latter two zones constituting the reaction zone 40. Usually, at its end opposite from the application zone 10, the device 1 includes an absorption zone 50 for enhancing diffusion of the liquid sample 3. The application zone 10 and the purification zone 20 may be the same zone. The diffusion medium 2 may be constituted by a single layer of a porous matrix, or more usually, by a plurality of layers of a porous matrix, each of the layers comprising one or more zones. Any type of material that is capable of ensuring that a fluid flows and is transferred may be used as the porous matrix. The fluid may be transferred by the force of capillarity. It is possible to use a bibulous material, e.g. of the filter paper membrane type, that absorbs liquid easily and through which the liquid is transported by capillarity. In general, such a device is placed in a cassette or box (not shown) that includes a well for depositing the sample in the application zone and a window for viewing the reaction zone.

The application (and purification) zone and/or the marking zone and/or the absorption zone may in particular be constituted by porous layers fitted on, or partially overlapping on, a first layer deposited over the entire surface of the support, or more usually a first layer deposited on a portion only of the surface of the support. This first layer deposited on the support serves as an analytic membrane and it incorporates the reaction zone (display zone and migration verification zone).

Thus, in order to facilitate fabrication of the device and enhance diffusion of deposited liquid sample (in the direction f₂ in FIG. 2), the various zones of the diffusion medium are constituted by a plurality of layers of a porous matrix that overlap and that are in fluid flow communication, as shown in FIG. 2B, which is described in detail when describing the examples. In particular, it is possible to use different layers of membranes or of filter paper. In particular, as shown in FIG. 2B, a layer corresponding to the purification zone 300, and also to the application zone, including activated carbon 301, may partially overlap a layer corresponding to the marking zone 500. The layer corresponding to the marking zone 500 can in turn overlap the analytic membrane 400 incorporating the display zone 600 and the migration verification zone 700. The analytic membrane 400 may extend over the entire surface of the support 100, as shown in FIG. 2B, or over a portion only of the support. Such overlaps are conventionally used in the state of the art (see in particular WO 2012/172232 and WO 2008/018073) and they make it possible in particular to ensure continuity of the flow of the liquid sample.

The difference between the device of the invention as shown in FIG. 2B and the prior art device as shown in FIG. 2A is the presence of activated carbon 301 in the zone 300.

In conventional manner, in the device of the invention, the first and second binding partners may be such as described above, and in particular antibodies or antibody fragments, and/or the diffusion medium may be a fiber material, in particular made of cellulose fibers or of glass fibers. Usually, the fiber material is made of nitrocellulose. By way of example, for the analytic membrane, it is possible to use a nitrocellulose fiber medium secured directly to a support, in particular of the polyester type.

Immunological reactions, i.e. the binding between antigens and binding partners, may be viewed by any detector means as a result of the first binding partner being marked with a marker. Thus, the first binding partner may bind marked particles that are capable of generating a detectable signal, e.g. including a marker, that may be in the form of a compound or a substance that can be detected by visual, fluorescence, or instrumental means. A non-limiting list of such markers are particles of metal or of an alloy, such as colloidal gold particles, particles of polymer, such as colored latex particles, magnetic particles, fluorescent molecules, chemiluminescent molecules, . . . . The signal generated in the result display zone and the signal generated in the migration verification zone, and that correspond to a positive result, may be of kinds that are identical or different. For example, when using colored latex, they may be of the same color or of different colors.

In a preferred implementation, the detection method of the invention uses a device as described in the present patent application.

The methods and devices of the invention for determining the presence of a microorganism in a patient are compatible with emergency situations. The symptoms of a gastroenteritis of viral origin last for only a few days, so the method and the device of the invention, when used in first intention, make it possible quickly to take the measures needed for rapidly protecting the patient and the patient's environment. The result can be interpreted within ten minutes of depositing the sample in the application zone. In care establishments, revealing norovirus by an immunochromatographic test using the method or the device of the invention leads to reinforcing hygiene measures, i.e. isolating the patient and accentuating disinfection of surfaces in order to avoid an epidemic. Furthermore, a positive result in the context of detecting a virus, makes it possible to set aside a bacterial infection, which might lead to hospitalizing elderly people, and can limit the use of antibiotics, which is pointless for a viral infection.

The examples below serve to illustrate the invention, but are of no limiting character.

### Example 1: Preparing an Immunochromatographic Device for Detecting Norovirus, the Device Including a Purification Zone Based on Activated Carbon

Preparing the Device

### Preparing Sample Pads

The sample pad is a strip of fiberglass having dimensions of 8 centimeters (cm)×1.7 cm that is cut from a membrane obtained from the supplier Alhstrom (Cat. No. Grade 8975, Helsinki, Finland). Prior to assembling immunochromatography strips, the sample pad is conventionally plunged into a bath of a saturation buffer.

In the control immunochromatography device (device: REF), the sample pad was plunged for 4 hours (h) in a bath having a buffer containing sugars and casein. It was then dried for 12 h to 18 h at 37° C.

In the immunochromatography device of the invention (device CARBON), the sample pad was initially subjected to the same treatment as the control immunochromatography device. Thereafter, it was plunged into a bath of a reagent based on activated carbon. The reagent based on activated carbon contained 0.65 g/L of Tris base, 6.83 g/L of Tris-HCl, 8.55 g/L of NaCl, 0.05% of Tween® 20, 1% of bovine serum albumin, and 10 g/L of activated carbon (Norit CN1, from Norit Nederland BV, Amerstoort, Netherlands). The pH was adjusted to 7.2 prior to adding the activated carbon. After 1 h, the sample pad was removed from the bath, placed on a grid, and stove-dried for 1 h at 37° C.

### Preparation of the Conjugate Pad, Marking Zone

400 nm particles of red PL-Latex Carbonyl HiDyenRed latex sold by the supplier Agilent Technologies (Cat. No. PL6104-614#) were coated by adsorption using a mixture of two immunoglobulin G (IgG) (anti-norovirus rabbit polyclonal antibodies, namely the antibodies 542 and 544 (bioMérieux). After incubating for 12 h to 18 h at ambient temperature, the particles were saturated with a casein-based buffer in order to avoid non-specific adsorptions. The particles were then spread over a fiberglass support (Cat. No. Grade 8975, Alhstrom Helsinki, Finland), which was dried overnight at 37° C.

### Preparation of the Analytic Membrane, Reaction Zone

Three mouse monoclonal anti-norovirus antigen antibodies, namely clones 1H3C3, 11H12, and 2A7 (bioMérieux) were mixed together and diluted in a PBS solution. The solution as prepared in this way was spread using a BIODOT (trade name) appliance on a membrane of nitrocellulose supported by polyester reinforcement (Unisart® CN 140 backed, Sartorius, Cat. No. 1UN14ER050020), thus constituting the display zone, referred to as the test line (T). An anti-rabbit IgG polyclonal antibody was diluted in a PBS buffer. That solution was spread over the migration verification zone, situated downstream from the display zone, and referred to as the verification line (C). The membrane was then stove-dried for 12 h to 18 h at 37° C., and then conserved in a sealed aluminum pouch with a dehydrating sachet in order to preserve it from moisture.

### Cassette Preparation

The strips were made by depositing the following on the nitrocellulose analytic membrane as supported in this way: a sample pad acting as a zone for applying the sample and for purifying it (fiberglass membrane acting as a filter for the sample before contact with the particles); the conjugate pad (marking zone); and an absorbent pad (an absorbent having the ability to adsorb the remainder of the sample after migrating along the various types of pad, and acting as an absorption zone). This pad was made using oil filter paper from the “automotive filter paper” range of the supplier Hangshou Xinhua Paper Industry Co. Ltd. (Hangzhou Tonglu, China). The assembly was then cut into strips having a width of 4 millimeters (mm).

The immunochromatography device of the invention (device: CARBON) is shown in FIG. 2B and differs from the device shown in FIG. 2A (device: REF) only by the fact that the first zone 300 acting both as a sample application zone and as a purification zone includes activated carbon 301. These devices enable the liquid sample to migrate in the direction f₂ and they include a multilayer diffusion medium 200. The multilayer diffusion medium 200 comprises an analytic membrane 400 with incorporated support 100 that forms the first layer and it includes various successive zones in the direction f₂: a first zone 300 for applying and purifying the sample and including the activated carbon 301; a marking zone 500 including at least a first marked binding partner; a display zone 600; and a migration verification zone 700, these latter two zones constituting the reaction zone 800. In certain locations, the diffusion medium is a multilayer medium. It should be observed that the layer corresponding to the application zone 300 on which the sample is deposited overlaps a layer that includes the marking zone 500. The layer including the marking zone 500 then overlaps the layer referred to as the analytic membrane 400, which includes the display zone 600 and the migration verification zone 700 for verifying flow continuity and for encouraging the liquid to diffuse by capillarity. At the end opposite from the zone 300, the device has an absorption zone 900 positioned on the layer 400 and serving to encourage diffusion of the sample.

Thereafter, these strips are individually sealed in plastics cassettes each having a sample well and a display window, ready for use.

### Operating Mode of the Immunochromatography Test

Remove the cassette from its sachet and place it on a surface that is clean and flat;
Deposit 75 microliters (μL) of the sample for testing in the sample well; and
Start a timer in order to read the test 10 min after deposition.

The status of the sample is determined as a function of the presence or absence of a colored line of variable intensity on the nitrocellulose membrane. The intensity of the colored line varies from L1 (no red line) to L10 (an intense red line is present). By comparing it with a read card, it is possible to make an objective evaluation of the intensity of the color of the colored line.

The results are interpreted in accordance with Table 1 below:

TABLE 1

| Reading | Results | Interpretation |
|---|---|---|
| Line of intensity < L4 | Negative | Looked-for analyte |
| | | absent |
| Line of intensity = | Positive, but | Looked-for analyte |
| L4 or L5 | close to the | is a priori |
| | detection limit | present |
| | of the kit | |
| Line of intensity > L5 | Positive | Looked-for analyte |
| | | is present |

### Detecting Norovirus by Immunochromatography

The stools are individually diluted in the VIKIA® Rota/Adeno diluant (Cat. No. 31111, bioMérieux) using 25 milligrams (mg) of stools in 1500 μL of diluant, and then vortexed for 15 s, with 75 μL being deposited in the well of the cassette. The results are given in Table 2 below.

TABLE 2

| | Stools positive for norovirus* | |
|---|---|---|
| Sample code | 87 | 437 |
| Device REF | L3 | L5 |
| Device CARBON | L4 | 16 |

*Stools of patients infected by norovirus.

The use of the immunochromatography device of the invention having a purification zone incorporating activated carbon serves to improve the detection of antigens in both of the samples selected above.

### Example 2: Using a Sampling Device with a Filter During the Pretreatment of the Stool Samples, and then Detecting Norovirus by Immunochromatography

Preparing the Stool Sampling Device with a Filter

The prototype was made from the sampling tube (tube with a green stopper) in the VIKIA® Rota/Adeno kit (Cat. No. 31111, bioMérieux). The white sampling wand contained in the green stopper was removed, a Whatman GF/B (1 μm filter) having a diameter of 6 mm (Cat. No. 1821110) was installed in the bottom of the stopper, and then the end of the wand (remote from the sample) was replaced in the stopper in order to hold the filter in place. The purpose of the filter was to retain the particles of carbon and to obtain a sample suitable for placing directly in the well of the cassette.

### Pretreatment of Stools and Immunochromatography

Three stool pretreatment methods were compared:
Method 1: REF

The stools were individually diluted in VIKIA® Rota/Adeno diluant (Cat. No. 31111, bioMérieux) at 50 mg of stools for 3000 μL of diluant. The stools were vortexed for 15 s. 75 μL of the sample as obtained in that way were deposited in the well of the immunochromatography cassette.
Method 2: CENTRIFUGING (treated with carbon+centrifuging) and Method 3: INVENTION

The reagent based on activated carbon as used in Example 1 was used again. The stools were individually diluted in the carbon reagent and 50 mg of stools per 3000 μL of diluant, and then:
Method 2: they were vortexed for 15 s and then centrifuged for 5 min at 12,000 g; and
Method 3: they were spread in the sampler device with the previously prepared filter.

In all of the methods, after breaking the nipple present at the end of the stopper, 75 μL of filtrate was deposited in the well of the cassette.

Table 3 below summarizes the conditions.

TABLE 3

| | | |
|---|---|---|
| Method 1 | Method 2 | Method 3 |

| Stools diluted using | | | Stools diluted with carbon reagent | | |
|---|---|---|---|---|---|
| VIKIA ® Rota/Adeno | | | | | |
| diluant (REF) | | | | | |
| VIKIA ® | 3000 | μL | Carbon reagent | 3000 | μL |
| Rota/Adeno | | | | | |
| diluant | | | | | |
| Stools | 50 | mg | Stools | 50 | mg |

| Vortex | 15 | seconds | Vortex | 15 | seconds | — | — |
|---|---|---|---|---|---|---|---|
| — | — | | Centrifuging | 5 | min | Filtering | Preparation |
| | | | | 12,000 | g | | spread |
| | | | | | | | in the |
| | | | | | | | prototype |
| Deposit | 75 | μL | Deposit | 75 | μL | Deposit | 75 μL |

In each method, the volume of the deposit was 75 μL and reading was performed 10 min after depositing the sample. The tests were carried out using a device REF as described in Example 1, and they were read and interpreted in the same manner as in Example 1. The results are given in Table 4 below.

TABLE 4

| | | | | Method 3 |
|---|---|---|---|---|
| | | | | INVENTION |
| | | | | Stools |
| | | Method 1 | Method 2 | diluted in |
| | | REF | CENTRIFUING | carbon |
| | | Stools | Stools | reagent + |
| | | diluted in | diluted in | filtering |
| | | VIKIA ® | carbon | 1 μm |
| | | Rota/Adeno | reagent + | filter |
| | | diluant | centrifuging | Diameter |
| | Stopper | Reference | Reference | 0.6 cm |
| Control | VIKIA ® | L1 | 11 | L1 |
| sample | Rota/Adeno | | | |
| | diluant | | | |
| Samples | E8909 GII.3 | L5 | L8 | L7-8 |
| positive for | E9982 GII.4 | L4 | L9 | L6 |
| norovirus* | E9918 GI.4 | L4 | L7 | L6-7 |
| | E8624 GI | L8 | L9 | L9 |

*stools of patients infected with norovirus.

The use of carbon does not degrade specificity.
Samples close to the detection limit (L4/L5) under the reference condition (Method 1) are very positive with the carbon treatment (Methods 2 and 3).
The intensity of the read strip is greater for samples treated with carbon.
For positive samples, when using carbon treatment, the test line appeared more quickly and the membrane was cleaner (the stools no longer color the membrane).
The results obtained by treating the samples with a carbon-based diluant confirm the improved sensitivity.
Against all expectations, the use of activated carbon, without a centrifuging step, makes it possible to obtain results that can be interpreted clinically in identical manner to the results obtained when a centrifuging step is used. In both methods, the results are positive (L≧6), whereas with the reference, the results were a priori positive. Separating by filtering instead of by centrifuging enables satisfactory sensitivity to be conserved.

1. A determination method for determining the presence of a target microorganism in a patient from a sample of said patient's stools, the method being characterized in that it comprises the following operations:
obtaining a sample of liquid stools of said patient or a liquid sample obtained from stools of said patient, referred to as the liquid sample;
pretreating the liquid sample with activated carbon; and
using immunochromatography, also known as lateral flow immunoassay, with an immunochromatography device having a sample application zone, a marking zone, and a reaction zone, to detect from the resulting pretreated liquid sample the possible presence of at least one antigen of the target microorganism so as to come to a conclusion about the presence or the absence of the target microorganism in said patient.

2. A determination method according to claim 1, characterized in that the pretreatment with activated carbon includes putting the liquid sample into contact with the activated carbon and separating the liquid sample as obtained thereby from the activated carbon.

3. A determination method according to claim 1, characterized in that it includes preparing the liquid sample of stools from said stools of said patient, and in that the pretreatment with the activated carbon is performed while preparing the liquid sample by mixing together stools, a liquid diluant, and activated carbon, followed by separating, enabling the liquid sample to be recovered without stools and without the activated carbon.

4. A determination method according to claim 1, characterized in that the pretreatment is performed by mixing the liquid sample with activated carbon followed by separating, enabling the liquid sample to be recovered without the activated carbon.

5. A determination method according to claim 1, characterized in that separating is performed by filtering.

6. A determination method according to claim 1, characterized in that the pretreatment is performed by passing the liquid sample over activated carbon positioned in a sampler device for taking the sample or in the immunochromatography device.

7. A determination method according to claim 1, characterized in that the liquid sample contains a diluant comprising a buffer, a denatured charge protein, and a detergent.

8. A determination method according to claim 1, characterized in that the microorganism is selected from viruses, bacteria, and parasites, and is preferably a rotavirus, an adenovirus, or more preferably a norovirus.

9. A determination method according to claim 1, characterized in that detection consists in detecting the interaction of at least one antigen for the microorganism of interest with at least one binding partner for binding to said at least one antigen, said binding partner preferably being an antibody or an antibody fragment.

10. A device (I, 1) for detecting at least one antigen of a target microorganism in a liquid sample (3), the device comprising:
a) a support (100); and
b) a porous diffusion medium (2, 200) fixed on the support (100), and enabling the liquid sample (3) to migrate, said diffusion medium (2, 200) comprising:
i) an application zone (10, 300) for applying the liquid sample (3);
ii) a purification zone (20, 300) including activated carbon (21, 301);
iii) a marking zone (30, 500) including at least a first marked binding partner, said first binding partner being capable of binding with said at least one antigen of the microorganism that is to be detected, if present in the liquid sample, then forming a first binding partner and antigen complex; and
iv) at least one reaction zone (40, 800) comprising:
a display zone (41, 600) for displaying the results of the detection and comprising at least one second binding partner held stationary on the diffusion medium and suitable for binding with said first binding partner and antigen complex;
a migration verification zone (42, 700) enabling proper operation of the device to be verified, which zone is situated downstream from the display zone (41, 600); and
said application zone (10, 300), purification zone (20, 300), marking zone (30, 500), and reaction zone (40, 800) being in communication to enable the liquid to diffuse.

11. A device (I, 1) according to claim 10, characterized in that the first and second binding partners are antibodies or antibody fragments and/or the diffusion medium is a fiber material, in particular made of cellulose fibers or of glass fibers.

12. A detection method according to claim 1, characterized in that it uses a device (I) according to claim 10.
